(19) United States
(12) Patent Application Publication
(10) Pub. No.: US 2005/0037371 A1
(43) Pub. Date: Feb. 17, 2005

(54) Systems and methods for sequence comparison

(76) Inventors: Jean-Jacques Codani, Rueil-Malmaison (FR); Guillaume Dufresne, Paris (FR); Manuel Duval, New London, CT (US); Eric Glemet, Courbevoie (FR); Hendrik Cornelis Heus, Rueil-Malmaison (FR); Laszlo Kristof Takacs, Newbury Park, CA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/723,522

(22) Filed: Nov. 26, 2003

Related U.S. Application Data
(60) Provisional application No. 60/429,965, filed on Nov. 29, 2002.

(51) Int. Cl. C12Q 1/68; G06F 19/00; G01N 33/48; G01N 33/50

(52) U.S. Cl. 435/6; 702/20

(57) ABSTRACT

Methods and systems for comparing a first sequence and a second sequence, including associating errors with alignments of the first sequence and the second sequence, comparing the alignment errors to identify the alignment having the smallest error, and, based on the alignment having the smallest error, computing: a first percent identity relative to the first sequence, and a second percent identity relative to the second sequence.

### CLAIM OF PRIORITY

This application claims priority to U.S. Ser. No. 60/429,965, entitled “Systems and Methods for Sequence Comparison”, filed on Nov. 29, 2002, the contents of which are herein incorporated by reference in their entirety.

### BACKGROUND

(1) Field

The disclosed methods and systems relate generally to pairwise alignment, and more particularly to computing percent identities based on pairwise alignments.

(2) Description of Relevant Art

Patent claims are generally directed to a nucleotide and/or polypeptide sequence invention and other sequences having a claimed level (e.g., percentage) of sequence identity to that invention. Freedom to operate and patentability assessments related to such claims can be based on queries of databases of such sequences, where such databases can include, for example, PAT 10, GENESEQ 5, EMBL, GenBank, and DDBJ. Unfortunately, search tools often do not identify sequences that may be within the scope of the patent claims, thus potentially causing an inaccurate evaluation and/or assessment of intellectual property rights.

Patentability and freedom-to-operate assessments can often rely on search and/or query tools that are based on homology. Such tools include, for example, BLAST (Basic Local Alignment Search Tool) and FASTA, which retrieve putative homologues based on a user-provided query sequence. Those of ordinary skill understand that two genes are homologues if they can be understood to have evolved from the same common ancestor gene. BLAST and FASTA can be understood to be directed to identifying a homology relationship between two sequences (e.g., the query sequence, and a database sequence), and accordingly, these tools are based on a set of parameters (e.g., gap opening penalty value, substitution matrix, cut-off scores, gap extension penalty, etc.) and a scoring system that conveys biological information. The configurable parameters can significantly alter the output of a given query, as the queries are based on an internally computed score that can be highly sensitive to these parametric changes. Examples can be provided where the same query may yield a 93% identity for one set of parameters, and a 100% identity with another set.

Particularly, sequence alignments may often be computed with percent identity scores only after the “best hits” are identified by a scoring function. As it's name implies, BLAST performs and optimizes an alignment with respect to a fraction of the query that gives the highest percentage score. Accordingly, the BLAST alignment can be achieved on a portion of the query, leading to a percent identity only with respect to a fraction of the query. Further, a user generally cannot specify which fraction of the query should be used for the alignment. BLAST can thus report multiple alignment results.

In contrast to BLAST, FASTA provides one alignment result by identifying local high scoring alignments, starting as a BLAST search with exact short word matches and extending potential hits based on a greedy ungapped basis. The result is a single gapped or ungapped alignment result.

Accordingly, regardless of whether BLAST and/or FASTA are employed, both query tools rely on the same homology-based search paradigm and the respective results are additionally based on upon a set of user-provided parameters that can significantly affect the query results. Searching or otherwise querying sequence databases based on patent claims 2 necessitates retrieving database elements that either completely match the query or are related to a specified extent, regardless of homology. The aforementioned tools can thus be considered ineffective for freedom to operate and patentability assessments.

### SUMMARY

The disclosed methods and systems include a method for comparing a first sequence and a second sequence, the method including associating errors with alignments of the first sequence and the second sequence, comparing the alignment errors to identify the alignment having the smallest error, and, based on the alignment having the smallest error, computing a first percent identity relative to the first sequence, and a second percent identity relative to the second sequence. The method can include determining a mismatch number based on mismatches between the first sequence and the second sequence based on the alignment having the smallest error, and/or an alignment number based on matches between the first sequence and the second sequence based on the alignment having the smallest error.

In an embodiment, computing a first percent identity relative to the first sequence can include determining an alignment number based on the matches between the first sequence and the second sequence based on the alignment having the smallest error, and, forming a ratio based on the alignment number and the length of the first sequence. Further, computing a second percent identity relative to the second sequence can include determining an alignment number based on the matches between the first sequence and the second sequence based on the alignment having the smallest error, and, forming a ratio based on the alignment number and the length of the second sequence.

In some embodiments, the methods and systems can include computing a third percent identity relative to the alignment having the smallest error. The third percent identity can be computed by determining an alignment number based on the matches between the first sequence and the second sequence based on the alignment having the smallest error, and, forming a ratio based on the alignment number and the length of the alignment. The matches can be perfect matches and/or positive matches. In an embodiment, a user or another can provide an input as to whether the percent identities can be computed based on perfect and/or positive matches.

In an embodiment, a user or another can provide a percent identity threshold, such that the methods and systems can include determining whether at least one of the first percent identity and the second percent identity is greater than a percent identity threshold.

In some embodiments where the methods and systems can include inserting gaps into either of the first and/or second sequence, the methods and systems can including determining a number based on the gaps in the first sequence based on the alignment having the smallest error, and, a number based on the gaps in the second sequence based on the alignment having the smallest error.

In one embodiment, one or more databases can be provided, where the database(s) can include one or more sequences, and the first and/or second sequences can be retrieved from the database(s). Accordingly, the disclosed methods and systems can allow for a single database of sequences to be compared against itself, and the methods and systems can also be extended to include comparisons of sequences from multiple databases. In one exemplary embodiment, the first sequence(s) can include one or more polypeptide sequence(s) and/or nucleotide sequence(s), and, the second sequence(s) can include one or more polypeptide sequence(s) and nucleotide sequence(s). Accordingly, the methods and systems can allow for serial and/or parallel processing of sequence alignment/comparison using one or more processing threads.

Aligning or otherwise comparing the first sequence and the second sequence can include aligning the first sequence and the second sequence, and computing an error based on the number of mismatches in the alignment. As provided previously herein, the alignment can include one or more insertion events with respect to the first sequence and/or the second sequence. In an embodiment, the alignment can be understood to include computing a string edit distance. In one embodiment, the string edit distance can be associated with the alignment error, and in such an embodiment, the alignment having the smallest error may be associated with the smallest string edit distance for a given first sequence and second sequence.

The disclosed methods and systems can include identifying whether the first sequence is longer than the second sequence, whether the second sequence is longer than the first sequence, and/or whether the first sequence and the second sequence are equivalent and/or equal length. In an embodiment where string edit distance can be determined, the query string can be understood to be the shorter sequence, and the target sequence can be understood to be the longer sequence. Alignment errors can be computed by or otherwise based on determining a string edit distance by computing those alignments where the shorter sequence can be included in (e.g., overlap) the longer sequence. When the first and second sequences are the same length, alignment errors can be computed based on the first sequence being the shorter sequence, and further, based on the second sequence being the shorter sequence. In one embodiment, the methods and systems can include aligning at least the entirety of the shorter sequence with at least a fragment of the longer sequence. Aligning at least the entirety can include inserting at least one gap into at least one of the shorter sequence and/or the longer sequence. In some embodiments, the alignment can be performed regardless of homology.

Alignment errors, including the alignment having the smallest error, can be compared to an alignment error threshold. A user or another can provide the alignment error threshold, and in some embodiments, an output may be based on the alignment error threshold such that alignments having a number of alignment errors exceeding the alignment error threshold may not be output or otherwise provided, for example.

A user or another can also provide a percent identity threshold, where outputs for the methods and systems can be based on a comparison of the first percent identity and the second percent identity relative to the percent identity threshold. In one example, if either of the first percent identity or the second percent identity exceeds the percent identity threshold, the methods and systems can output (e.g., transmit to display, memory, storage, another application, another device, and/or otherwise provide) data (e.g., first percent identity, second percent identity, third percent identity, scoring matrix(s), scoring matrix(s) metrics (e.g., perfect matches, positive matches, etc.), alignment error data, number of gaps in first sequence, number of gaps in second sequence, alignment identification data, positions of alignments, etc.). Those of ordinary skill can recognize that the methods and systems can include comparing the length of the first sequence with the length of the second sequence, and performing the alignments based on the length comparison and a percent identity threshold. Accordingly, if the length difference(s) between a first and second sequence exceeds a percent identity threshold, alignments may not be performed for the first and second sequence pair.

In an embodiment, the aligning can be performed based on a dynamic programming method for approximate string (e.g., sequence) matching. For example, the methods and systems can include determining locations at which a query string/sequence (e.g., first sequence) of length m matches a sub-string (e.g., subsequence) of a subject string/sequence (e.g., second sequence) of length n, where n is longer than m, and where such locations of alignment can provide less than k errors. K can be an alignment error threshold.

The methods and systems can include one or more interfaces to allow a user or another to identify the first sequence(s) (e.g., database(s)), identify the second sequence(s) (e.g., database(s)), provide a percent identity threshold, and/or provide an alignment error threshold. Accordingly, the methods and systems can include performing multiple sequence comparisons, and can thus include, iteratively, storing the first percent identity and the second percent identity, retrieving a first sequence and/or a second sequence, and repeating the process of associating errors with the alignments, to provide at least one stored first percent identity and second percent identity, where such stored percent identities can exceed a percent identity threshold, and can be associated with alignments having a number of alignment errors less than or equal to an alignment error threshold. The stored percent identities can be associated with the first and second sequences to which they apply, and such storage can be sorted based on, for example, percent identity.

Other objects and advantages will become apparent hereinafter in view of the specification and drawings.

### BRIEF DESCRIPTION OF THE DRAWINGS

FIGS. 1A and 1B depict illustrative embodiments of the disclosed systems and methods;

FIG. 2 is an exemplary block diagram providing some components of an illustrative system and method;

FIGS. 3A-3C show local alignment, global alignment, and best-fit alignments; and,

FIG. 4 is an example scoring matrix.

### DESCRIPTION

To provide an overall understanding, certain illustrative embodiments will now be described; however, it will be understood by one of ordinary skill in the art that the systems and methods described herein can be adapted and modified to provide systems and methods for other suitable applications and that other additions and modifications can be made without departing from the scope of the systems and methods described herein.

Unless otherwise specified, the illustrated embodiments can be understood as providing exemplary features of varying detail of certain embodiments, and therefore, unless otherwise specified, features, components, modules, and/or aspects of the illustrations can be otherwise combined, separated, interchanged, and/or rearranged without departing from the disclosed systems or methods. Additionally, the shapes and sizes of components are also exemplary and unless otherwise specified, can be altered without affecting the scope of the disclosed and exemplary systems or methods of the present disclosure.

The disclosed methods and systems include methods and systems to compare two sequences that include a first sequence and a second sequence. In one embodiment, the sequences can be polypeptide and/or nucleotide sequences, although the methods and systems are not so limited, and other sequences of ASCII characters can be used, where such sequences can apply to other applications and/or embodiments. Generally, references to the word “sequence” herein can be understood to be an ASCII string. The methods and systems can be used to compare the lengths of the first and second sequences to determine a shorter sequence and a longer sequence, and to determine a best fit of the shorter sequence in the longer sequence. Based at least on the best fit, a first percent identity can be computed relative to the first sequence, and a second percent identity can be computed relative to the second sequence. The first and second percent identities can be provided as an output to a display, a database, a memory, a computer program, another processor-controlled device, and/or another output device. In one embodiment, where the first and second sequences can be based on polypeptide and/or nucleotide sequences, the methods and systems can be employed to determine whether the first and second sequences may be within the scope of a patent claim that may be associated with one or both of the first sequence and the second sequence.

In one embodiment, a first sequence can be a sequence provided in and/or otherwise proposed for inclusion in a patent application, while the second sequence(s) can be one or more prior art sequence, and thus, the methods and systems may be applied to the first and second sequence(s) to determine whether the first sequence is novel when compared to the second sequence(s), and/or whether an entity is free to use such first sequence (e.g., a freedom to operate analysis as is known in the art). As provided herein, the first and/or second sequence can be understood more generally to be a string, such as a biological sequence and/or a synthetic sequence, with such examples provided for illustration and not limitation.

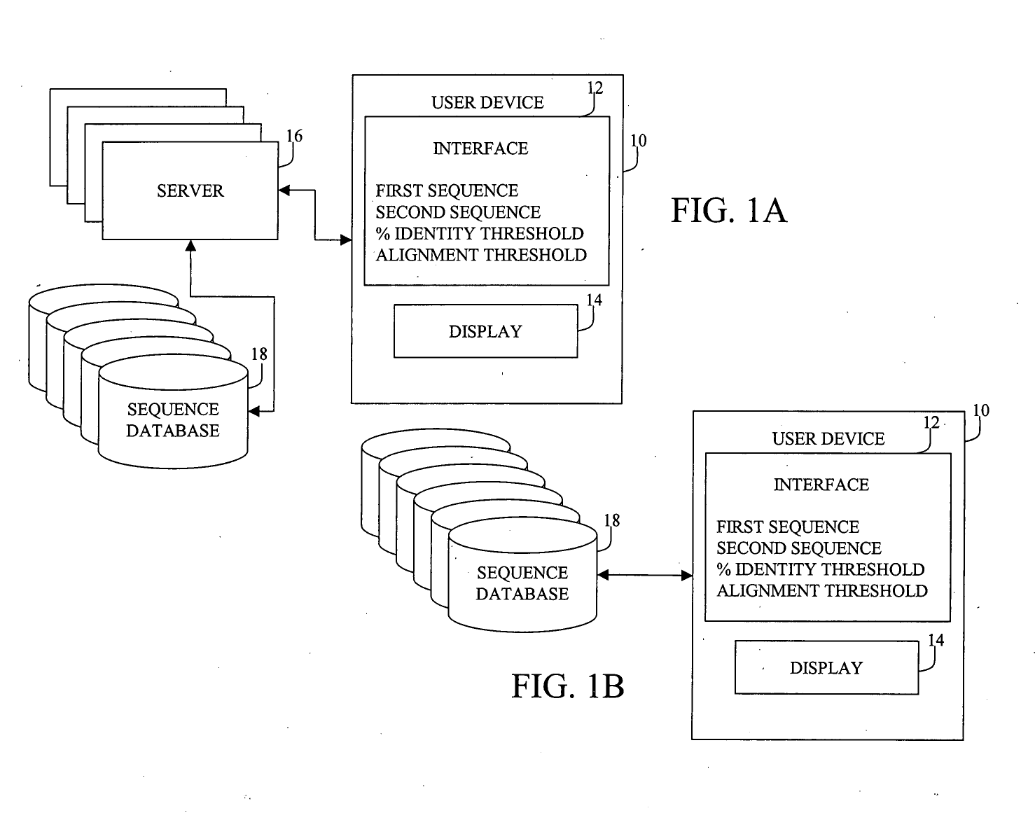

FIG. 1A shows one illustrative embodiment for implementing the disclosed methods and systems. As FIG. 1 indicates, a user-device 10, which can be a processor-controlled device as provided herein, can be provided with a user-interface 12 that can allow a user or another to input information and/or data that can be used by the disclosed methods and systems. As FIG. 1A shows, such information can include an identity of a first sequence and an identity of a second sequence. Those of ordinary skill will understand that in one embodiment, the first sequence can be, for example, a “query” sequence, while the second sequence can be and/or include one or more databases which can include or otherwise contain one or more “target” sequences to which the first/query sequence can be compared. In other embodiments, the first sequence and the second sequence can be individual sequences, and the first sequence can be associated with the target, while the second sequence can represent the query. In one embodiment, the first sequence can be a database and/or otherwise associated therewith, and the second sequence can be a database and/or otherwise associated therewith, and in some instances, such databases can be the same to indicate a request for performing the methods and systems against a single database. In other embodiments, the databases can be different. Accordingly, those of ordinary skill will recognize that references herein to first sequence and second sequence can include one or more identifiers for one or more query sequences, and/or one or more identifiers for one or more target sequences, such as one or more databases of target sequences. Such identifiers can be provided via a user input or other designation means.

Accordingly, the designations and/or references to first and second sequences herein can be understood to be arbitrary, and for the discussion herein, it can be understood that the first sequence is the query sequence (or a reference and/or identifier related thereto, e.g., one or more databases), while the second sequence is the target sequence (or a reference and/or identifier related thereto, e.g., one or more databases).

Referring again to FIG. 1A, in some embodiments, a user or another can provide, via a user interface or other means (e.g., specified by a system manager, hard-coded) a percent identity threshold which can indicate a percent identity of comparison between the first and second sequences, such that the computed percent identities relative to the first sequence and the second sequence can be compared to the percent identity threshold. In one embodiment, for example, computed percent identities may be stored when one or both of such computed percent identities equal or exceed the threshold. Other comparisons to the threshold can be made.

FIG. 1A also indicates that the user or another can enter, via a user interface or another means, an alignment error threshold that can be used, for example, for comparison against a number of alignment errors between two sequences. In an embodiment, if a number of errors between the two sequences (first sequence and second sequence) equal or exceed the alignment error threshold, such alignment may not be deemed to be desirable by the user, may not be output by the system, and/or may not otherwise be included in computations, where such examples are provided for illustration and not limitation.

Those of ordinary skill will recognize that the use of a percent identity threshold and an alignment error threshold can be optional, and as provided herein previously, although in some embodiments, such thresholds can be variable and/or user-specified, in other embodiments, the thresholds may be fixed by, for example, a system administrator, user, or another.

As indicated by FIG. 1A, the user entered information can be provided to one or more servers 16, where such servers 16 can be understood to be associated with one or more processor controlled devices as provided herein. Such servers 16 can include instructions for accepting the user-provided information and for accessing processor-executable instructions as provided herein for providing and/or otherwise performing sequence comparisons/alignments. The servers 16 can have access to one or more databases 18 which can include databases of sequences such as polypeptide and/or nucleotide sequences, although other sequences can be included, and such sequences can include alphanumeric sequences, binary sequences, and/or other strings. In an embodiment according to FIG. 1A, the user can request a comparison by providing the aforementioned user-specified information at a user device 10, where such information can be transmitted to a server(s) 16 via a wired or wireless connection using one or more intranets and/or the internet, where the servers 16 can thereafter process the request by accessing the databases 18. Such database accessing can include querying the databases 18 based on the user information. Upon completing the requested sequence comparisons, the servers 16 can provide the user-device 10 with outputs and/or results that can be provided to a memory, the device display 14, or other location.

Those of ordinary skill in the art will recognize that the FIG. 1A illustrative system can be understood to be representative of a client-server paradigm, where the instructions on the user device 10 for obtaining user information and requesting a comparison can be a client, and the servers 16 can be a server in the client-server paradigm. Accordingly, it can be understood that the illustrated user device 10 instructions and instructions on the servers 16 can be included in a single device such as provided by FIG. 1B, where such embodiment may also be considered within the client-server paradigm. As FIG. 1B indicates, the user device 10 can access, via wired or wireless communications and using one or more intranets and/or the internet, the databases 18 for querying and/or retrieving sequences. Additionally, the FIG. 1B embodiment can represent an embodiment that may not include a client-server paradigm.

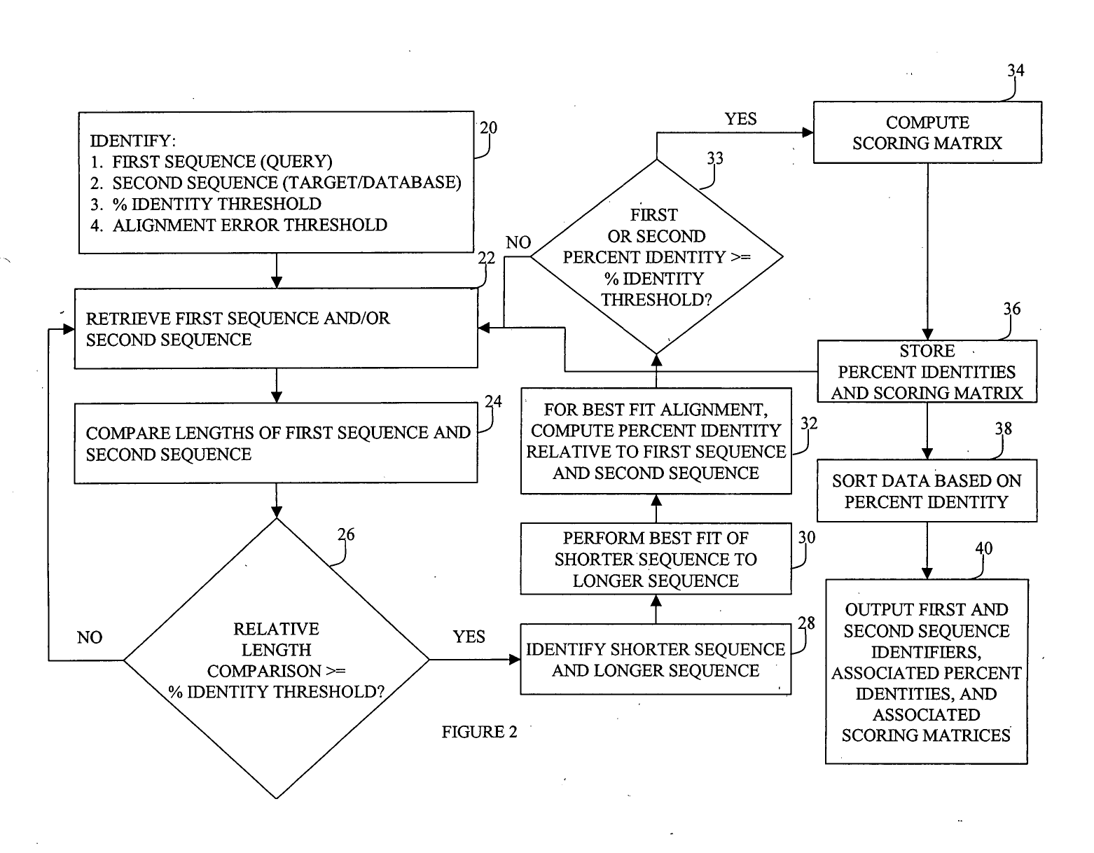

Referring to FIG. 2, there is one illustrative block diagram for one embodiment of the disclosed methods and systems. As FIG. 2 indicates, using a user device 10 or another means, a user or another can provide an identifier to identify at least one first sequence, at least one second sequence, and optionally, a percent identity threshold, and an alignment error threshold 20. For the purposes of discussion with respect to the illustrative embodiments, reference can be made to a single first sequence and a single second sequence, although it can be understood as provided previously herein, that the methods and systems can be applied to one or more first sequences, one or more second sequences, where such sequences can be in a single and/or multiple databases, and thus such discussion is merely for convenience and can be understood to encompass or otherwise embody multiple first and/or second sequences.

Referring again to FIG. 2, the first and second sequences can be retrieved or otherwise provided, obtained, and/or identified 22, whereupon the lengths of the sequences can be determined and compared 24. In one embodiment, such as the embodiment according to FIG. 2, for example, if the comparison of the lengths is less than the percent identity threshold 26, no further processing may be performed, and the disclosed method and system can retrieve another first sequence and/or second sequence 22 as provided by the embodiment. For example, if the percent identity threshold is ninety-percent, the exemplary embodiment requests alignments equal to or exceeding such percent identity threshold, and the first sequence is length ten, while the second sequence is length one-thousand, one of ordinary skill in the art will recognize that such length comparison can indicate that such percent threshold cannot be satisfied using an alignment, and thus, determining an alignment may not be performed. Such a decision 26 can be optional, as may other decisions in other illustrative embodiments.

According to the FIG. 2 embodiment, when the relative and/or comparative sequence lengths equals and/or exceeds the percent identity threshold 26, one of the first or second sequences can be identified as the shorter (“query”) sequence, while the other such sequence can be identified as the longer (“target”) sequence 28. In embodiments when the first sequence and the second sequence may be the same length, the methods and systems disclosed herein can be applied to such sequences with the first sequence identified as the shorter sequence and the second sequence identified as the longer sequence, and again, with the first sequence identified as the longer sequence and the second sequence identified as the shorter sequence. As provided previously herein, a best-fit module can be applied to the sequences 30 to determine and/or identify at least one fragment of the longer sequence which can provide a best-fit 33 for at least the entirety of the shorter sequence. The best-fit can be understood to include one or more alignments. The best fit can be determined based on computing a string edit distance, which can be computed in one or more manners, and such computation can additionally and/or optionally include other dynamic programming methods and systems.

Accordingly, in one embodiment, the disclosed methods and systems can be understood to include computing, determining, or otherwise providing an edit distance between two strings and/or sequences (“string edit distance”), where the string edit distance can be understood to be a minimum number of character inserts (“insertion event”) and/or changes to convert a first string/sequence, to a second string/sequence, where the second sequence can be greater than or equal in length to the first sequence. The insertion events can occur in either the first string and/or the second string. For example, given a first sequence of length m (“pattern” sequence), and a second sequence of length n, informally, the string/sequence edit distance can include computing the smallest edit distance between the first sequence and sub-strings of the second sequence. A sub-string matching method and/or system can thus be understood to identify a best-fit position of a given sub-string (e.g., shorter of the first sequence and the second sequence) within another longer, string (e.g., longer of the first sequence and the second sequence). In one embodiment, a result can include the beginning position within the target (longer) string/sequence where the best match (e.g., minimum number of errors between the sequences) is found.

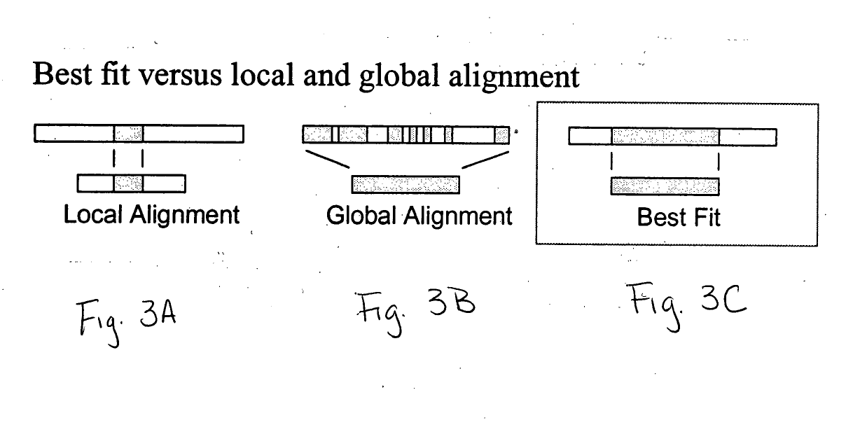

Those of ordinary skill will recognize that the “best-fit” methods and systems can be distinguished from local alignment methods and systems that may, for example, align only a portion (e.g., sub-sequence) of the shorter (“query”) sequence/string, and similarly, can be distinguished from global alignment methods and systems that may, for example, attempt to align the entirety of the shorter (“query”) sequence/string to the entirety of the longer (“target”) sequence/string. The disclosed methods and systems, as provided herein, attempt to align at least the entirety of the shorter sequence to at least a fragment of the longer sequence. Those of ordinary skill will understand that aligning at least the entirety of the shorter sequence may include an alignment such that a portion of the shorter sequence aligns with at least a fragment of the longer sequence, where such at least a fragment of the longer sequence may include one or both of the ends of the longer sequence. An example of the local alignment, global alignment, and best-fit alignment is shown in FIGS. 3A-C, respectively.

Referring again to FIG. 2, for at least the alignment having a smallest string edit distance compared to the other alignments and associated string edit distances (“alignment having the smallest error”), the methods and systems can at least compute a first percent identity relative to the first sequence and a second percent identity relative to the second sequence 32. Such computations can include determining an alignment number, where the alignment number can be based on the matches between the first sequence and the second sequence, and such matches can be based on the alignment having the smallest error. Those with ordinary skill will understand that such alignment can include “gaps” in the first sequence and/or the second sequence, and thus the length of the alignment can be longer than, for example, the shorter sequence, as such alignment can include gaps in the shorter sequence.

In one embodiment, the disclosed methods and systems can compute an alignment number based on the number of perfect matches in the alignment, and/or the number of positive matches in the alignment. Those of ordinary skill will understand a positive alignment to include an acceptable substitution, such as when the first and second sequences include amino acids, and one or more amino acids can mutate into another amino acid to allow a positive, rather than a perfect, match. In an embodiment, a user or another can provide an input as to whether the percent identities can be computed based on perfect and/or positive matches.

The disclosed methods and systems can thus include computing percent identities based on the number of perfect matches relative to the length of the first sequence, the length of the second sequence, and/or the length of the alignment having the smallest error. The methods and systems can also include computing percent identities based on the number of positive matches relative to the length of the first sequence, the length of the second sequence, and/or the length of the alignment having the smallest error. The methods and systems can include computing percent identities based on the number of perfect and positive matches relative to the length of the first sequence, the length of the second sequence, and/or the length of the alignment having the smallest error. Those of ordinary skill will recognize that a percent identity can include forming a ratio based on one or more of the aforementioned alignment numbers (e.g., negative, perfect, positive matches), and a length of one or more of the different sequences.

Accordingly, although not shown in the FIG. 2 embodiment, as provided herein, a third percent identity can be computed by determining an alignment number based on the matches between the first sequence and the second sequence based on the alignment having the smallest error, and, forming a ratio based on the alignment number and the length of the alignment.

In an embodiment not shown in FIG. 2, a decision may be additionally and/or optionally based on the aforementioned alignment error threshold. Accordingly, the methods and systems can include, for example, determining a number (e.g., an error) based on the number of mismatches based on the alignment having the smallest error, and if such number of mismatches exceeds the alignment error threshold, the methods and systems may return to retrieving another first and/or second sequence 22.

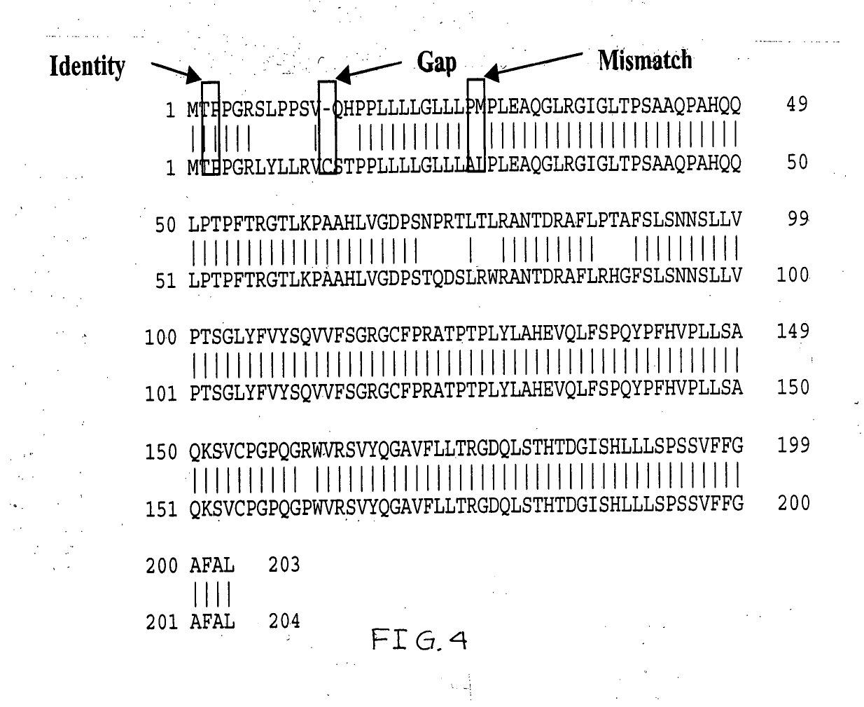

Referring again to FIG. 2, based on the percent identities computed relative to the first and second sequences, and whether at least one of such percent identities equals or exceeds the percent identity threshold, and/or whether an alignment number is less than or equal to an alignment error threshold, a scoring matrix can be computed 34 for the at least one best-fit alignment. Those of ordinary skill will recognize that scoring matrices can include the first sequence, the second sequence, and indicators identifying, for each element in the respective sequence, whether there is a perfect match (“|”), a positive match (“+”), or a negative match (“ ”). Such indicators are exemplary. The scoring matrix can be based on the alignment have the smallest error. One example of a scoring matrix is shown in FIG. 4.

The disclosed methods and systems can include computing a number based on the gaps in the first sequence, and a number based on the gaps in the second sequence, for an alignment having a smallest error. Further, those of ordinary skill will understand that a “best-fit” alignment may include multiple alignments for a given first and second sequence, where such multiple “best-fit” alignments thus provide different alignment results.

In the FIG. 2 embodiment, the scoring matrix data provided herein (e.g., scoring matrix, number of “perfect”, “positive”, and “negative” matches (e.g., “mismatch”), number of gaps in first and/or second sequence, etc., and the aforementioned percent identities can be optionally stored 36 and the methods and systems can retrieve another first and/or second sequence 22. The alignment and/or comparison data that is stored can be optionally sorted 38 and/or otherwise organized and output 40 to a user or another, to provide one or more data files that can include graphics and/or other data, where such output data can be transmitted 40 via wired and/or wireless networks to, for example, the user device 10, a display, a memory, another processor-controlled device, and/or another specified location. The output data can be based on one or more of the aforementioned quantities (e.g., percent identities, numbers, scoring matrix metrics, etc.) and/or data items (e.g., scoring matrix), although the illustrated embodiment of FIG. 2 includes a subset of such output data. In one embodiment, a user can select output formats and/or locations, and can input data, for example, to indicate whether a scoring matrix data and/or metrics can be computed and/or output.

The methods and systems described herein are not limited to a particular hardware or software configuration, and may find applicability in many computing or processing environments. The methods and systems can be implemented in hardware or software, or a combination of hardware and software. The methods and systems can be implemented in one or more computer programs, where a computer program can be understood to include one or more processor executable instructions. The computer program(s) can execute on one or more programmable processors, and can be stored on one or more storage medium readable by the processor (including volatile and non-volatile memory and/or storage elements), one or more input devices, and/or one or more output devices. The processor thus can access one or more input devices to obtain input data, and can access one or more output devices to communicate output data. The input and/or output devices can include one or more of the following: Random Access Memory (RAM), Redundant Array of Independent Disks (RAID), floppy drive, CD, DVD, magnetic disk, internal hard drive, external hard drive, memory stick, or other storage device capable of being accessed by a processor as provided herein, where such aforementioned examples are not exhaustive, and are for illustration and not limitation.

The computer program(s) can be implemented using one or more high level procedural or object-oriented programming languages to communicate with a computer system; however, the program(s) can be implemented in assembly or machine language, if desired. The language can be compiled or interpreted.

As provided herein, the processor(s) can thus be embedded in one or more devices that can be operated independently or together in a networked environment, where the network can include, for example, a Local Area Network (LAN), wide area network (WAN), and/or can include an intranet and/or the internet and/or another network. The network(s) can be wired or wireless or a combination thereof and can use one or more communications protocols to facilitate communications between the different processors. The processors can be configured for distributed processing and can utilize, in some embodiments, a client-server model as needed. Accordingly, the methods and systems can utilize multiple processors and/or processor devices, and the processor instructions can be divided amongst such single or multiple processor/devices.

The device(s) or computer systems that integrate with the processor(s) can include, for example, a personal computer(s), workstation (e.g., Sun, HP), personal digital assistant (PDA), handheld device such as cellular telephone, laptop, handheld, or another device capable of being integrated with a processor(s) that can operate as provided herein. Accordingly, the devices provided herein are not exhaustive and are provided for illustration and not limitation.

References to “a microprocessor” and “a processor”, or “the microprocessor” and “the processor,” can be understood to include one or more microprocessors that can communicate in a stand-alone and/or a distributed environment(s), and can thus can be configured to communicate via wired or wireless communications with other processors, where such one or more processor can be configured to operate on one or more processor-controlled devices that can be similar or different devices. Use of such “microprocessor” or “processor” terminology can thus also be understood to include a central processing unit, an arithmetic logic unit, an application-specific integrated circuit (IC), and/or a task engine, with such examples provided for illustration and not limitation.

Furthermore, references to memory, unless otherwise specified, can include one or more processor-readable and accessible memory elements and/or components that can be internal to the processor-controlled device, external to the processor-controlled device, and/or can be accessed via a wired or wireless network using a variety of communications protocols, and unless otherwise specified, can be arranged to include a combination of external and internal memory devices, where such memory can be contiguous and/or partitioned based on the application. Accordingly, references to a database can be understood to include one or more memory associations, where such references can include commercially available database products (e.g., SQL, Informix, Oracle) and also proprietary databases, and may also include other structures for associating memory such as links, queues, graphs, trees, with such structures provided for illustration and not limitation.

References to a network, unless provided otherwise, can include one or more intranets and/or the internet. References herein to microprocessor instructions or microprocessor-executable instructions, in accordance with the above, can be understood to include programmable hardware.

Unless otherwise stated, use of the word “substantially” can be construed to include a precise relationship, condition, arrangement, orientation, and/or other characteristic, and deviations thereof as understood by one of ordinary skill in the art, to the extent that such deviations do not materially affect the disclosed methods and systems.

Throughout the entirety of the present disclosure, use of the articles “a” or “an” to modify a noun can be understood to be used for convenience and to include one, or more than one of the modified noun, unless otherwise specifically stated.

Elements, components, modules, and/or parts thereof that are described and/or otherwise portrayed through the figures to communicate with, be associated with, and/or be based on, something else, can be understood to so communicate, be associated with, and or be based on in a direct and/or indirect manner, unless otherwise stipulated herein.

Although the methods and systems have been described relative to a specific embodiment thereof, they are not so limited. Obviously many modifications and variations may become apparent in light of the above teachings. Many additional changes in the details, materials, and arrangement of parts, herein described and illustrated, can be made by those skilled in the art. Accordingly, it will be understood that the following claims are not to be limited to the embodiments disclosed herein, can include practices otherwise than specifically described, and are to be interpreted as broadly as allowed under the law.

1. A method for comparing a first sequence and a second sequence, the method comprising:
associating errors with alignments of the first sequence and the second sequence,
comparing the alignment errors to identify the alignment having the smallest error, and,
based on the alignment having the smallest error, computing: a first percent identity relative to the first sequence, and a second percent identity relative to the second sequence.

2. A method according to claim 1, further including determining at least one of:
a mismatch number based on mismatches between the first sequence and the second sequence based on the alignment having the smallest error, and,
an alignment number based on matches between the first sequence and the second sequence based on the alignment having the smallest error.

3. A method according to claim 2, where:
the mismatches are negative matches, and,
the matches can be at least one of perfect matches and positive matches.

4. A method according to claim 1, where computing a first percent identity relative to the first sequence includes:
determining an alignment number based on the matches between the first sequence and the second sequence based on the alignment having the smallest error, and,
forming a ratio based on the alignment number and the length of the first sequence.

5. A method according to claim 4, where:
the mismatches are negative matches, and,
the matches can be at least one of perfect matches and positive matches.

6. A method according to claim 1, where computing a second percent identity relative to the second sequence includes:
determining an alignment number based on the matches between the first sequence and the second sequence based on the alignment having the smallest error, and,
forming a ratio based on the alignment number and the length of the second sequence.

7. A method according to claim 6, where:
the mismatches are negative matches, and,
the matches can be at least one of perfect matches and positive matches.

8. A method according to claim 1, further including computing a third percent identity relative to the alignment having the smallest error.

9. A method according to claim 8, where computing a third percent identity includes:
determining an alignment number based on the matches between the first sequence and the second sequence based on the alignment having the smallest error, and,
forming a ratio based on the alignment number and the length of the alignment.

10. A method according to claim 1, further including,
determining whether at least one of the first percent identity and the second percent identity is greater than a percent identity threshold.

11. A method according to claim 10, further including providing a percent identity threshold.

12. A method according to claim 1, further including determining at least one of:
a number based on the gaps in the first sequence based on the alignment having the smallest error, and,
a number based on the gaps in the second sequence based on the alignment having the smallest error.

13. A method according to claim 1, further including:
providing at least one database, the at least one database including at least one sequence, and,
retrieving at least one of the first sequence and the second sequence from the at least one database.

14. A method according to claim 1, where,
the first sequence includes at least one of: at least one polypeptide sequence and at least one nucleotide sequence, and,
the second sequence includes at least one of: at least one polypeptide sequence and at least one nucleotide sequence.

15. A method according to claim 1, where associating errors with alignments includes,
aligning the first sequence and the second sequence, and,
computing an error based on the number of mismatches in the alignment.

16. A method according to claim 1, where associating errors with alignments includes,
aligning the first sequence with the second sequence based on at least one insertion event in at least one of: the first sequence and the second sequence.

17. A method according to claim 1, where associating errors includes computing a string edit distance.

18. A method according to claim 1, where associating errors includes comparing a number of alignment errors to an alignment error threshold.

19. A method according to claim 1, where associating errors with alignments includes,
comparing a length of the first sequence to a length of the second sequence to identify a shorter sequence and a longer sequence, and,
aligning at least the entirety of the shorter sequence with at least a fragment of the longer sequence.

20. A method according to claim 19, where aligning at least the entirety includes inserting at least one gap into at least one of the shorter sequence and the longer sequence.

21. A method according to claim 19, where comparing includes,
determining that the first sequence length is equal to the second sequence length, and,
associating the first sequence with the shorter sequence and the second sequence with the longer sequence, and performing the aligning, and,
associating the first sequence with the longer sequence and the second sequence with the shorter sequence, and performing the aligning.

22. A method according to claim 19, where comparing includes,
determining that the first sequence length is equal to the second sequence length, and,
associating at least one of:
the first sequence with the shorter sequence and the second sequence with the longer sequence, and,
the first sequence with the longer sequence and the second sequence with the shorter sequence.

23. A method according to claim 1, where associating errors includes aligning regardless of homology.

24. A method according to claim 1, where associating errors includes performing at least one pairwise alignment.

25. A method according to claim 1, where associating errors includes implementing a dynamic programming module for approximate string matching.

26. A method according to claim 1, further including:
comparing the length of the first sequence with the length of the second sequence, and,
performing the alignments based on the length comparison and a percent identity threshold.

27. A method according to claim 1, further including providing at least one interface to perform at least one of: identify the first sequence, identify the second sequence, provide a percent identity threshold, and provide an alignment error threshold.

28. A method according to claim 1, further comprising outputting the first percent identity and the second percent identity.

29. A method according to claim 1, further comprising outputting the first percent identity and the second percent identity based on at least one of: a percent identity threshold and an alignment error threshold.

30. A method according to claim 1, further comprising outputting a scoring matrix associated with the first percent identity and the second percent identity.

31. A method according to claim 1, further comprising outputting data based on a comparison of the first percent identity and the second percent identity with a percent identity threshold.

32. A method according to claim 1, further comprising:
iteratively,
storing the first percent identity and the second percent identity,
retrieving at least one of a first sequence and a second sequence, and,
returning to associating errors,
to provide at least one stored first percent identity and second percent identity.

33. A method according to claim 32, where storing includes associating the first percent identity and the second percent identity with at least one of the first sequence and the second sequence.

34. A method according to claim 32, further comprising:
sorting the at least one stored first percent identity and second percent identity based on percent identity, and,
outputting the sorted first percent identity and second percent identity.

34. A method according to claim 1, further comprising:
performing in at least one parallel processing thread,
storing the first percent identity and the second percent identity, and,
retrieving at least one of a first sequence and a second sequence, and,
returning to associating errors,
to provide at least one stored first percent identity and second percent identity.

35. A method according to claim 1, where at least one of the first sequence and the second sequence includes an ASCII string.

36. A method according to claim 1, where at least one of the first sequence and the second sequence includes an identifier to a database of sequences.

37. A method according to claim 1, where the first sequence includes an identifier to a first database and the second sequence includes an identifier to a second database of sequences.

38. A method according to claim 37, where the first database and the second database are the same.


SEQUENCE LISTING (2 sequences)

SEQ ID NO: 1 - protein, 203 aa, Bos taurus
MTPPGRSLPPSVQHPPLLLLGLLLPMPLEAQGLRGIGLTPSAAQPAHQQLPTPFTRGTLKPAAHLVGDPSNPRTLTLRANTDRAFLPTAFSLSNNSLLVPTSGLYFVYSQVVFSGRGCFPRATPTPLYLAHEVQLFSPQYPFHVPLLSAQKSVCPGPQGRWVRSVYQGAVFLLTRGDQLSTHTDGISHLLLSPSSVFFGAFAL

SEQ ID NO: 2 - protein, 204 aa, Bos taurus
MTPPGRLYLLRVCSTPPLLLLGLLLALPLEAQGLRGIGLTPSAAQPAHQQLPTPFTRGTLKPAAHLVGDPSTQDSLRWRANTDRAFLRHGFSLSNNSLLVPTSGLYFVYSQVVFSGRGCFPRATPTPLYLAHEVQLFSPQYPFHVPLLSAQKSVCPGPQGPWVRSVYQGAVFLLTRGDQLSTHTDGISHLLLSPSSVFFGAFAL